(19) United States
(12) Patent Application Publication
(10) Pub. No.: US 2011/0300551 A1
(43) Pub. Date: Dec. 8, 2011

(54) METHOD OF PREDICTING CLINICAL OUTCOMES FOR MELANOMA PATIENTS USING CIRCULATING MELANOMA CELLS IN BLOOD

(76) Inventors: Galla Chandra Rao, Princeton Junction, NJ (US); Mark C. Connelly, Doylestown, PA (US)

(21) Appl. No.: 13/155,687

(22) Filed: Jun. 8, 2011

Related U.S. Application Data
(60) Provisional application No. 61/352,441, filed on Jun. 8, 2010.

(51) Int. Cl. C12Q 1/68; G01N 33/574; C12Q 1/06

(52) U.S. Cl. 435/6.14; 435/39; 435/7.23

(57) ABSTRACT

The present invention provides an automated method for capturing and detecting circulating melanoma cells (CMC's) in the blood of patients with melanoma. The absolute number of circulating melanoma cells detected in the peripheral blood tumor load is, in part, a factor in prediction of survival, time to progression, and response to therapy.

### BACKGROUND

Treatment of advanced melanoma is complicated by its heterogeneous histopathology and changes in make-up that accumulates during tumor progression. The enumeration and characterization of circulating tumor cells in patients with either metastatic breast or colorectal cancer has been shown to provide independent prognostic and predictive information that is clinically significant and can be used to monitor patient management.

Circulating tumor cells (CTC's) have been shown to be a critical link between primary cancer, a disease stage at which cure is possible, and metastatic disease, which continues to be the leading cause of death for most malignancies. Clinical studies have shown that CTC's are a powerful prognostic and predictive biomarker in metastatic breast cancer, and similar findings have been reported in prostate cancer and colorectal cancer. These data show that CTC's are representative of the underlying biology driving metastatic cancer and suggest that further cellular and molecular analyses of these cells can reveal new insights into molecular regulation of metastasis and response to therapy.

Methods to capture, enumerate, and characterize CTCs have been modified to capture enumerate and characterize circulating melanoma cells (CMCs) in a patient's blood. See Automated Enumeration and Characterization of Circulating Melanoma Cells in Blood, U.S. patent application Ser. No. 12/254,188, filed Oct. 20, 2008. This application is hereby incorporated by reference. Even though CMCs were captured, enumerated and characterized by this method, the predictive value, with respect to the short term survival of patients with metastatic melanoma was unknown. This invention offers a method of predicting overall survival for patients with metastatic melanoma.

### BRIEF DESCRIPTION OF THE DRAWINGS

FIG. 1. Recovery of known numbers of spiked SK-Mel 28 cells from whole blood. SK-Mel28 cells spiked into the healthy donor samples (i.e., 0, 5, 18, 72, 280 and 1183 cells were spiked into 7.5 mL of blood from five healthy donors on each of 2 days with a total of 5 different samples at each cell level. The number of cells spiked is plotted versus the observed number of cells recovered.

FIG. 2. Rows A-E represent objects that were identified by the CellTracks Analyzer II® software as objects having both DAPI and PE signal in a sample from a melanoma patient. From right to left the thumbnail images represent the Ki67 FITC signal, the CD45 and or CD34 APC signal, the DAPI signal, the HMW-MAA PE signal and the overlay of DAP1 (purple) and HMW-MAA (green) signal. The cell in Row A is excluded as a melanoma cell as it expresses CD45 and or CD34, the cell in Rows B and C are classified as melanoma cells that do not express Ki67 and the cell in Rows D and E are classified as melanoma cells that do express Ki67.

FIG. 3. Gallery of typical CMC images from the CellTracks Analyzer II® obtained from 7.5 mL of blood from melanoma patients.

FIG. 4. Prevalence of CMC in 7.5 mL of blood of 55 healthy donors, 79 samples from 44 metastatic melanoma patients. Panel A and the percentage of Ki67 expressing CMC in 19 samples from 16 melanoma patients, Panel B.

FIG. 5. Kaplan-Meier estimates of probabilities of Overall Survival in patients with metastatic melanoma for those with <2 Circulating Melanoma Cells per 7.5 ml of whole blood and those in the group with ≧2 Circulating Melanoma Cells in 7.5 ml of whole blood. OS times were calculated from the time of each blood draw. Median survival is 12.1 months for the group with <2 CMC versus 2.0 months for people with ≧2 CMC (P=0.001 by the log-rank test; hazard ratio of death in patients with ≧2 cells per 7.5 ml, 3.2).

### DETAILED DESCRIPTION OF THE INVENTION

The invention includes a method of predicting overall survival for patients with metastatic melanoma comprising:
a) obtaining a 7.5 mL blood sample from a patient with metastatic melanoma, said sample comprising a mixed cell population suspected of containing circulating melanoma cells;
b) enriching a fraction of said specimen, said fraction containing said circulating melanoma cells;
c) confirming structural integrity of said rare cells to be intact;
d) analyzing said intact rare cells; wherein said analyzing correlates disease progression;
e) evaluating the number of circulating melanoma cells in said blood sample
   wherein if the number is greater than or equal to 2 predicting that the patient's overall survival will be low, and
   wherein if the number of circulating melanoma cells is less than two, predicting that the patient's overall survival will be high.

As used herein the term “enriching” means isolating CMCs from the blood sample of step (a). Methods of enriching include but are not limited to using anti CD146 coupled to magnetic particles. The preferred method is using antibodies to antigens present on melanoma cells coupled to magnetic beads to capture cells from the blood sample. The term “confirming” means determining whether the isolated cells are CMCs or other cellular components. Methods confirming include but are not limited to using a nucleic acid dye or a monoclonal antibody specific for melanoma cells. The preferred method of confirming is staining the CMCs with different fluorescently labeled monoclonal antibodies and the preferred antibodies are CD45 & CD34 to exclude leukocytes and endothelial cells, and high molecular weight melanoma associated antigen, HMW-MAA to identify melanoma cells. The term “analyzing” means evaluating the captured CMCs to determine if the CMCs express a variety of melanoma specific markers such as HMW-MAA, MART-1 (Melanoma antigen recognized by T-cells) and other markers such as Ki-67. The preferred method of analyzing means determining if the CMCs express Ki-67 and/or HMW-MAA. The term “evaluating” means determining how many CMCs are in the sample and using methods which include but are not limited to automated image analysis. The preferred method evaluating is using CellTracks Analyzer II®.

The invention is demonstrated by the following methods and examples. These examples and methods are not intended to limit the scope of the invention.

### EXAMPLES

### The Following Methods are Provided to Facilitate the Practice of the Present Inventions

Patients and Blood Collection. Blood was drawn from healthy volunteers and patients with malignant melanoma into evacuated 10-mL blood CellSave preservative blood draw tube (Veridex LLC, Raritan, N.J.) and processed within 72 hours.

The patients were all enrolled from the Department of Medical Oncology of the University of Oxford at the Churchill Hospital using a research ethics committee approved protocol. All patients provided written informed consent. Forty-four patients were enrolled, 25 males and 19 females, and their age ranged from 31-81 (mean 59). At the time of first blood draw 39/44 (86%) had metastatic disease and 5 patients had unresected stage III disease. 38/44 (78%) of patients with metastatic disease had visceral disease, 5/44 (11%) had no visceral involvement and for 1 patient the metastatic sites were not recorded. Median duration of follow up was 10.1 months. Blood was always drawn from cancer patients either before or a minimum of 7 days after the administration of intravenous therapy. Fifty-five healthy volunteers were included as controls and had no known illness or fever at the time of draw and no history of malignant disease.

Cell Culture and Cell Spiking. The melanoma cell line SK-Mel28 was cultured in flasks containing RPMI 1640 supplemented with 10% fetal calf serum and subsequently harvested without trypsinization. The cell suspensions were only used when their viability as assessed by trypan blue exclusion exceeded 90%. To determine the actual cell number, 200 μL of buffer and 20 μL of fluorescent beads (Beckman-Coulter. Inc., Miami, Fla.) containing approximately 20,000 total beads were added to a 504 aliquot of the SK-Mel28 cells. The SK-Mel28 cells were stained with anti HMW-MAA conjugated to PE for the detection. Duplicate tubes containing beads only were run on a flow cytometer (FACSCalibur; BD Biosciences, San Jose, Calif.) until 100% of the sample was aspirated. This provided an accurate estimate of the number of beads present in 20 μL. The experimental tubes were then tested in triplicate on the flow cytometer until 10,000 beads were counted in each tube. The number of SK-Mel28 cells was determined using the known number of beads per unit volume.

Sample Preparation. 7.5 mL of blood is transferred to 15 mL CellTracks® AutoPrep® sample tubes and mixed with 6.5 mL of buffer, centrifuged at 800 g for 10 minutes, and then placed on the CellTracksAutoprep® (Veridex LLC) for automated sample preparation. Reagents were optimized for capture and detection of melanoma cells and consisted of ferrofluids coated with CD146 antibodies to immunomagnetically enrich both melanoma cells and endothelial cells, a capture enhancement reagent to maximize the capture efficiency, a phycoerythrin-conjugated antibody that binds to the High Molecular Weight Melanoma Associate Antigen (HMW-MAA) (clone 9.2.27, Veridex LLC) to identify melanoma cells, a mixture of two allophycocyanine conjugated antibodies to identify leukocytes (CD45, clone HI30, Veridex LLC) and endothelial cells (CD34, clone 581, BD Biosciences), a FITC conjugated antibody identifying the Ki-67 protein (clone B56, BD Biosciences, San Jose, Calif.), the nuclear dye 4′,6-diamidino-2-phenylindole (DAPI) to identify nucleated cells and buffers to wash, permeabilize, and resuspend the cells. In the final processing step, the cells were resuspended in the MagNest® Cell Presentation Device (Veridex LLC). The magnetic field generated by the MagNest device causes the magnetically labeled cells to distribute uniformly over the analysis surface of the cartridge, ready for analysis using the CellTracks Analyzer II®.

Sample Analysis. The MagNest is placed on the CellTracks AnalyzerII®, a four-color semi-automated fluorescence microscope. Image frames covering the entire surface of the cartridge for each of the four fluorescent filter cubes are captured. Images that contain PE as well as DAPI positive events are presented in a gallery for classification of the events by the user based on cell fluorescence and morphology. The criteria for an object to be defined as a melanoma cell include round to oval morphology, a visible nucleus (DAPI positive), positive staining for HMW-MAA and negative staining for CD45 and CD34. The melanoma cells were divided in KI67+ and Ki67− cells. Results of cell enumeration are always expressed as the number of cells per 7.5 mL of blood.

Accuracy, Sensitivity, and Linearity of Melanoma Cell Detection. For accuracy, linearity, and sensitivity experiments, SK-Mel28 cells were spiked into 7.5 mL of blood collected into CellSave Preservative Tubes at 6 different levels of cells (0, 5, 18, 72, 280 and 1183). The exact number of cells spiked into blood was determined by flowcytometry. The samples were processed 24 hours after spiking the blood on a CellTracks AutoPrep® and analyzed with a CellTracks Analyzer II®. Sample testing was performed over two different days with a total of 5 different samples at each cell level.

Statistical Analysis

The primary endpoint was overall survival, measured as the time from the sample date to date of death from any cause. Patients who were lost to follow-up or still alive at the end of study were censored at the last date they were known to be alive or at the end of study date. If there were multiple samples per patient, the last sample was used for survival analysis. Overall survival was calculated using the Kaplan-Meier method and a survival plot was generated. Cox regression models was used to determine hazard ratios (HR) of death. Results were analyzed in SPSS 16.0 (SPSS Inc. Chicago. Ill., USA).

### Example 1

### Recovery of Spiked Tissue Culture Melanoma Cell Line (SK-Mel28)

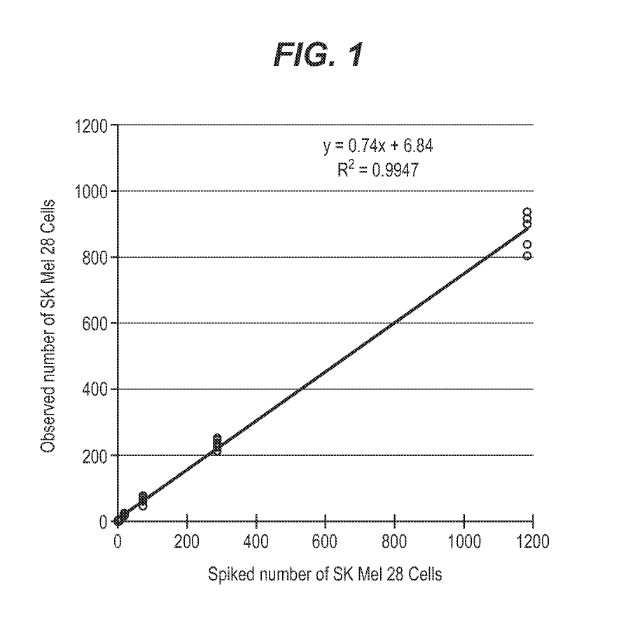

In this example, the assay performance using whole blood spiked with SK-Mel28 cells is described. The protocol used for this study was as follows. Whole blood was drawn into CellSave Tubes from healthy volunteers and spiked with tissue culture melanoma SK-Mel28 cells. Varying numbers of SK-Mel28 cells were spiked into blood, and recovery was measured. The expected number of SK-Mel28 cells spiked into the healthy donor samples (i.e., 0, 5, 18, 72, 280 and 1183 cells) plotted against the actual number of SK-Mel28 cells observed in the samples is shown in FIG. 1, and results are summarized in Table 1. Mean recovery of spiked cells was 88%, with recovery of 74% at the highest spike versus 88% at the 5 SK-Mel28 spike. Pearson R² correlation was 0.99. As expected, the coefficient of variation (CV) increased as the number of cells spiked decreased, ranging from 7% at the 1,183-cell spike to 31% at the 5-cell spike. The recovery of SK-Mel28 cells ranged from 64-120% and did not decrease with lower cell numbers.

TABLE 1
Method accuracy measured by recovery of SK-Mel 28 cells
spiked into 7.5 mL blood of five healthy donors

| Expected | Observed CMC Count | | | % Recovery | | |
|---|---|---|---|---|---|---|
| CMC count | Average | SD | 95% CI | Average | 95% CI | % CV |
| 0 | 0 | 0 | 0 | 0 | 0 | 0 |
| 5 | 4 | 1 | 3-5 | 88 | 64-112 | 31 |
| 18 | 20 | 2 | 18-22 | 110 | 100-120 | 10 |
| 72 | 63 | 11 | 53-73 | 87 | 74-100 | 17 |
| 287 | 234 | 15 | 221-247 | 81 | 77-85 | 6 |
| 1183 | 880 | 56 | 831-929 | 74 | 70-78 | 7 |

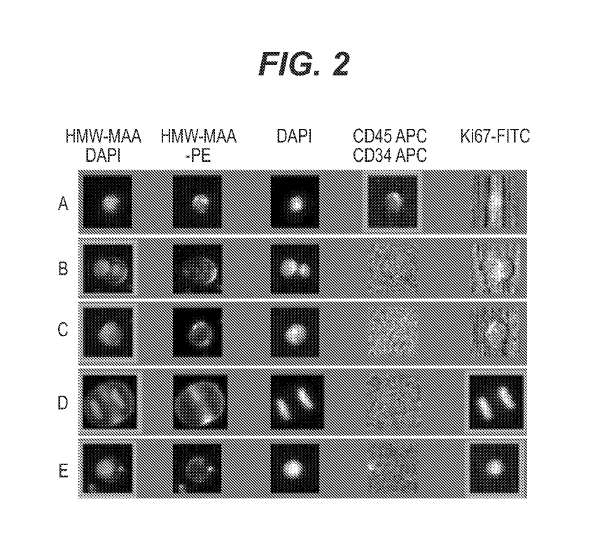

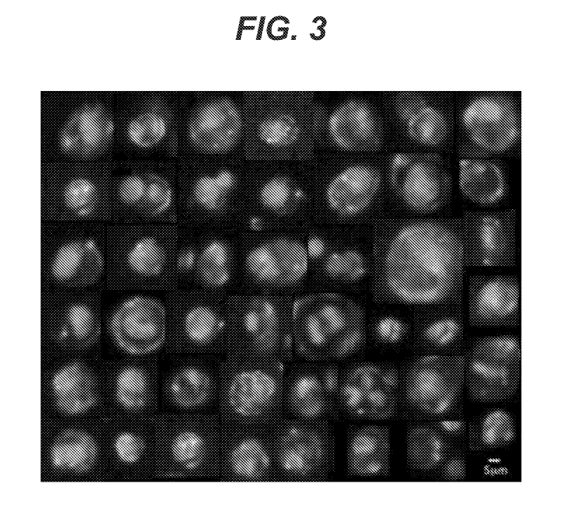

Identification of Circulating Melanoma Cells Thumbnail images of an overlay of HMW-MAA PE and HMW-MAA PE, DAPI, CD45/CD34 APC and Ki67 are presented to the operator for review. The presence of a nucleus, expression of HMW-MAA, cellular morphology, and a lack of CD45 or CD34 expression are the required characteristics of CMC. FIG. 2 shows 6 events from one melanoma patient that are presented to the reviewer. Panel A shows a cell staining with DAPI and HMW-MAA but also with CD34 and or CD45 and is thus not classified as CMC. Panels B, C, D and E show cells staining with DAPI and HMW-MAA but not with CD34 or CD45 and are classified as CMC. The CMC in Panels B and C do not express Ki67 whereas the CMC in Panels D and E do. Note that the CMC in Panel B contains two nuclei and does not stain with Ki67 whereas the CMC in Panel D appears to be actively dividing and indeed and indeed expresses Ki67. The size of the CMC and their nuclear to cytoplasmic ratio vary greatly between CMC within and between melanoma patients. FIG. 3 shows a gallery of CMC images from different patients with characteristically a round to oval shape and an intact nucleus. Cellular sizes varied over a wide range from 4 μm to 30 μm. Small cell clusters and multinucleated CMC, were also observed.

### Example 2

### Frequency of Circulating Melanoma Cells in Healthy Volunteers and Melanoma Patients

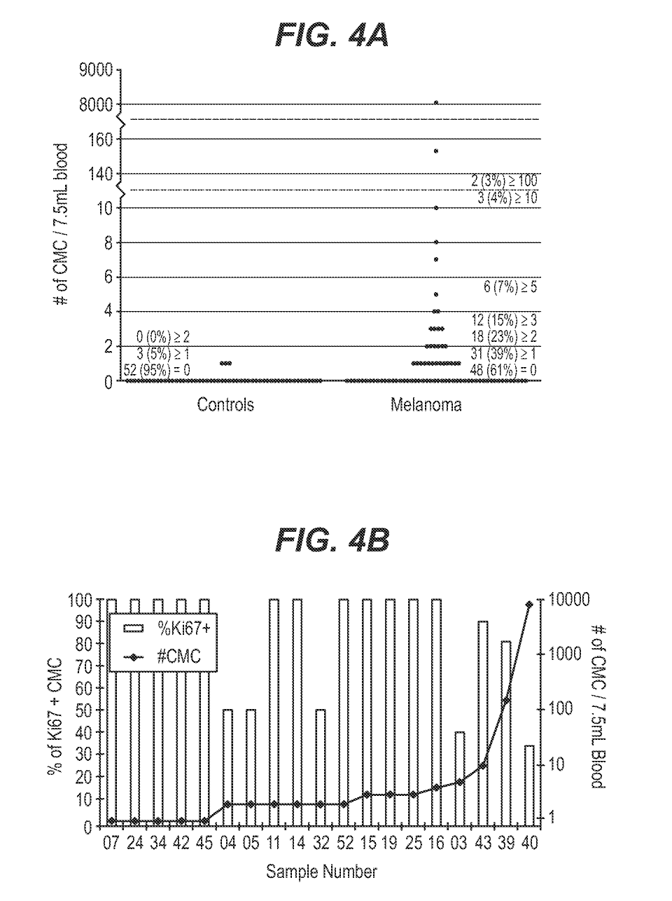

In this example, the frequency of circulating melanoma cells in healthy volunteers and melanoma patients is described. CMC were enumerated in 55 blood samples from healthy donor and 79 samples from 44 patients with metastatic melanoma. FIG. 4, Panel A shows the number of CMC detected in 7.5 mL of blood of the control group and the patients. Assessment of Ki67 expression was determined in 19 samples from 17 patients in whom CMC were detected. The percentage of Ki67+CMC ranged from 34 to 100% with a mean of 84% (SD25). Panel B of FIG. 4 shows the Ki67 expression and the number of CMC detected in these samples. In the 55 healthy donors three cells were classified as CMC and all three did not express Ki67.

### Example 3

### Circulating Melanoma Cells and Overall Survival in Melanoma Patients

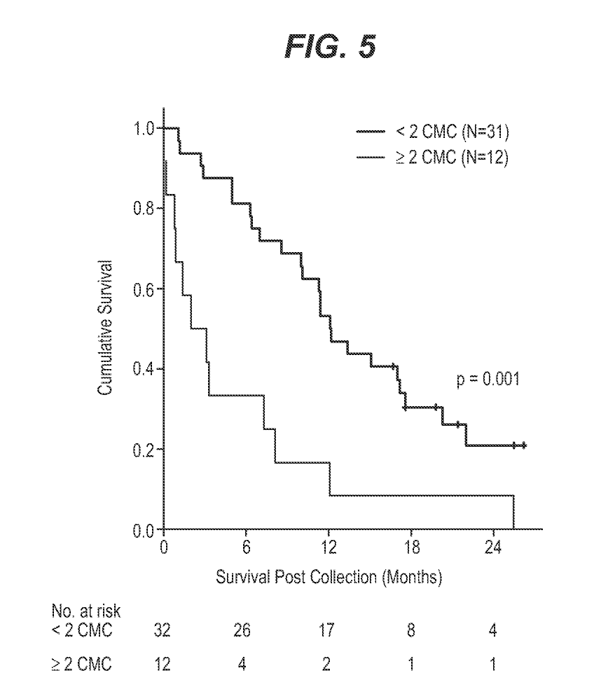

None of the individuals in the control group had 2 or more CMC detected and this cut-off was chosen to discriminate between patient groups. Mean OS time for those patients with <2 CMC was 12.1 months (95% CI 9.7. to 14.4) and was significantly longer than the median OS time for those patients with ≧2 CMC, 2.0 months (95% CI 0. to 4.9) (FIG. 5). Logrank p was 0.001. Hazard ratio of death was 3.2 (95% CI 1.6-6.5) by Cox Regression. The four patients that died within 1 month after blood draw had relatively high numbers of CMC (2, 8, 10 and 8043 CMC/7.5 ml).

What is claimed is:

1. A method of predicting overall survival for patients with metastatic melanoma comprising:
(a) obtaining a 7.5 mL blood sample from a patient with metastatic melanoma, said sample comprising a mixed cell population suspected of containing circulating melanoma cells;
(b) enriching a fraction of said specimen, said fraction containing said circulating melanoma cells;
(c) confirming structural integrity of said rare cells to be intact;
(d) analyzing said intact rare cells; wherein said analyzing correlates disease progression;
(e) evaluating the number of circulating melanoma cells in said blood sample
wherein if the number is greater than or equal to 2 predicting that the patient's overall survival will be low, and
wherein if the number of circulating melanoma cells is less than two, predicting that the patient's overall survival will be high.

2. A method as claimed in claim 1, wherein said fraction is obtained by immunomagnetic enrichment using an externally applied magnetic field to separate paramagnetic particles coupled to a biospecific ligand which specifically binds to said melanoma cells, to the substantial exclusion of other populations.

3. A method as claimed in claim 2, wherein said biospecific ligand is melanoma cell adhesion molecule CD146.

4. A method as claimed in claim 1, wherein said structural integrity is determined by a procedure selected from the group consisting of immunocytochemical procedures, FISH procedures, flowcytometry procedures, image cytometry procedures, and combinations thereof.

5. A method as claimed in claim 1, wherein said structural integrity is determined by a nucleic acid dye, a monoclonal antibody specific for High Molecular Weight Melanoma Associated Antigen.

6. The method as claimed in claim 5, wherein said structural integrity is further confirmed by exclusion of co-enriched leukocytes and circulating endothelial cells using leukocyte and endothelial specific antibodies.

7. The method of claim 6, wherein said specific antibodies are CD45 and CD34.

8. The method as claimed in claim 5 further containing CD45 and CD34 to exclude co-enriched leukocytes and circulating endothelial cells.

9. The method as claimed in claim 1, wherein FITC labeled anti-Ki67 is added to determine the proportion of CMC's in active cell cycle within the circulation.

10. The method of claim 1 wherein low overall survival is no more than two months.

11. The method of claim 1 wherein high overall survival is twelve months
